(19) United States
(12) Patent Application Publication
(10) Pub. No.: US 2014/0135659 A1
(43) Pub. Date: May 15, 2014

(54) MEASURING DEVICE FOR DETECTING POSITIONAL CHANGES OF PERSONS IN BEDS

(71) Applicants: Andrin Maggi, Zurich (CH); Michael Sauter, Neftenbach (CH)

(72) Inventors: Andrin Maggi, Zurich (CH); Michael Sauter, Neftenbach (CH)

(73) Assignee: Compliant Concept AG, Dubendorf (CH)

(21) Appl. No.: 14/130,396

(22) PCT Filed: Jun. 29, 2012
(86) PCT No.: PCT/CH2012/000146

§ 371 Date: Dec. 31, 2013

(30) Foreign Application Priority Data
Jul. 1, 2011 (CH) 1112/11

(51) Int. Cl. A61B 5/00; A61B 5/11

(52) U.S. Cl. CPC A61B 5/447; A61B 5/1116; A61B 5/1126; A61B 5/6891
USPC 600/595

(57) ABSTRACT

A measuring device for detecting positional changes of persons in a bed, having a bridge (10), which is arranged in a middle region of the lying surface transversely to the bed and is supported there by at least two supporting regions spaced apart from each other. The bridge (10) supports a mattress lying on the lying surface (1). The measuring device includes a sensor (13, 13a) at at least one of the supporting regions. Through use of the measured values of the sensors (13, 13a) a controller (21) detects the center of gravity position of the weight distribution loading the bridge (10) and activates an alarm if the movement activity of a person lying on the bed is insufficient.

### BACKGROUND

The subject matter of the invention relates to a measuring device for detecting positional changes of persons in beds, a bed with a measuring device, and a method for detecting measurement variables in the case of a bed.

If a person lies down for a relatively long time without moving, pressure ulcers can appear at the pressure points of the body under load. In so doing, the skin and the tissue situated therebelow are damaged locally. Such pressure ulcers are also referred to as decubitus, decubitus ulcers or bedsores.

In healthy persons, the risk of bedsores is low because they often change their position even while sleeping. By contrast, in the case of people requiring care, the ability to change the position independently may be adversely affected or impaired. Therefore, they generally need to be monitored by care staff and repositioned regularly. Monitoring and repositioning persons at risk by care staff looking after them is complicated and expensive. The risk potential of bedsores is often estimated incorrectly. In the case of overworked and/or insufficiently trained care staff, there therefore is the risk of a person requiring care suffering from bedsores. This in turn leads to follow-on costs in the care of these persons, which, in the case of suitable preventive measures, could have been avoided.

German patent application DE4240782A1 discloses a method and a device for bedsore prophylaxis, wherein pressure values of body parts of a patient are measured. Here, the person lies on a soft-elastic mat with a multiplicity of pressure sensors, which are arranged in a grid structure. The mat is large enough so that at least the body region between the shoulders and the buttocks lies on the measuring area. The mat is smooth, can be adapted well to the body shape and is made from washable and/or sterilizable or disinfectable plastics. The sensors are connected to a computer or notebook for evaluating the measurement data by means of analog/digital transducers. This computer or notebook evaluates the pressure data from the sensors and generates an optical or acoustic alarm if the pressure load on the monitored person is too large and the person has to be re-bedded or repositioned. The multiplicity of sensors enable a differentiated detection of the pressure distribution within a relatively large measurement area. However, the evaluation of the sensor signals is relatively complicated. For effective bedsore prophylaxis, precise knowledge of local pressure points and the pressure distribution over a majority of the lying zone are not mandatory. In principle, it would suffice to detect positional changes of a person, which bring about sufficient unloading of pressure points at risk. Since the person to be monitored has to lie substantially directly on the mat, the effectiveness of optionally present other means applicable to the situation which are arranged below the mat for mounting and/or monitoring the person—i.e., for example, in or adjoining a mattress—may be adversely affected. Moreover, the mat must meet high hygiene standards. Cleaning and disinfection cause additional maintenance effort. Furthermore, the multiplicity of measuring lines between the sensors and the A/D-transducer can interfere during operation and when the mat is being cleaned.

### SUMMARY

An object of the present invention therefore lies in developing a measuring device for beds, which renders it possible to detect and evaluate positional changes of persons in these beds in a simple manner. The term “bed” comprises any lying devices with a mattress or any other corresponding support for lying on. This includes, in particular, care beds and beds in which a mattress lies on a lying area such as e.g. on a slatted frame, and in which this lying area is supported by a bed frame.

A further object of the invention is provided in developing the measuring device in such a way that it is mobile and can also be retro-fit to existing beds and that the complexity for the installation and maintenance thereof is as low as possible.

A further object of the invention is provided in developing a bed with a measuring device for bedsore prophylaxis, wherein this measuring device does not, where possible, adversely affect other functions of the bed.

A further object of the invention is provided in developing a method for processing measurement variables of the measuring device, by means of which it is possible to identify and signal instances of repositioning of a person lying on the bed, which are required to prevent bedsores.

These objects are achieved by a measuring device and a bed in accordance with one or more of the features of the invention, and by a method for processing measurement variables according to the invention.

The invention is based on the discovery that the risk of bedsores in the case of a person lying in a bed can be assessed largely due to regular positional changes, particularly in the pelvic region of this person, or due to changes in the position of the center of gravity of the weight force acting on the mattress in the region of the buttocks or the pelvis. Positional changes of the pelvic region are accompanied by movements or changes in the contact pressure of other body parts, which are likewise at risk. If the person autonomously changes the position of their pelvis or the weight distribution in the pelvic area a number of times within a predetermined period of time, the risk of bedsores is low. Thus, in order to identify the risk of a pressure ulcer in a timely manner, it is sufficient to monitor movements or positional changes in the pelvic region of a person. This can be achieved by virtue of, in a central region of the lying area of a bed, the position of the center of gravity of the load supported by this central region being detected transversely to the longitudinal direction of the lying area. The pelvic region of the person to be monitored is supported in this central region of the lying area. If the person moves, this changes the distribution of the weight load transversely to the bed and hence also the position of the center of gravity in this central region. In order to detect the position of the center of gravity transversely to the bed, a bridge structure is arranged in a central region of the lying area between the head region and the foot region thereof, which bridge structure supports the mattress in the pelvic region of a person lying on said mattress. In a first embodiment, the bridge structure—a lso abbreviated to bridge in the following—comprises a rigid transverse slat or an elongate plate, the length of which at least approximately corresponds to the width of the bed in which it is to be used. At least in the region of its two ends, the plate comprises support points or support regions which are suitable for supporting, carrying, mounting or holding on the bed frame or generally on a support device. Depending on the embodiment of the bridge and of the bed, the support can take place e.g. directly on longitudinal struts of a bed frame formed to the side of the lying area. The plate can also be supported indirectly on the frame by means of connection means. Such connection means can comprise elements of the bed frame and/or elements of the measuring device. In particular, cross struts of the bed frame or slats of a slatted frame can be employed as connection means.

In a particularly advantageous embodiment of the invention, the bridge comprises a further plate with higher bending elasticity at a short distance below the rigid plate. The upper, rigid plate is supported at the two shorter ends by means of support bodies or spacers on the lower bearing plate, at least in the region of the support points. Sensors for detecting the locally supported weight force can be formed in the region of the support points on the plate itself, on the support bodies or on the bearing plate arranged therebelow or, in general, on connection means. As a result of its bending elasticity, the bearing plate can be supported in the case of different support structures or bed frames. The support bodies are borne by the lower plate even if a gap or depression in the bed frame is situated directly below. The weight applying a load to the bridge can be reliably transmitted to the two longitudinal struts of the bed frame in any bed, with the sensors supplying information about the distribution of the weight load to the two longitudinal struts.

The bridge preferably comprises a washable, removable sheath made of plastic, in which the two plates are embedded. The controller for evaluating the sensor signals can be arranged partly or wholly e.g. between the two plates within the sheath.

At least part of the controller with analog/digital transducers for digitizing the sensor signals is preferably arranged in the region of the bridge, for example between the two plates. As a result, the influence of interference signals can be minimized. The electronics in the interior of the sheath can be connected to a part of the controller arranged outside of the bridge by means of a connection cable guided to the outside. Alternatively, use can also be made of a wireless connection for communication between a control part within the sheath and an external control part. If a rechargeable battery is additionally arranged in the region of the bridge, which, for example, can be recharged contactlessly by means of an inductive charging device, the sheath can optionally be hermetically sealed. From the measurement variables of the sensors, the controller can derive changes in the weight distribution applying the load to the plate or, in general, the bridge or transverse connection.

In a preferred embodiment, the bridge comprises one or more resistive force or pressure sensors, in particular strain gauges, also abbreviated DMS in German, which change the electric resistance thereof depending on the respective pressure or tension load. Alternatively, use can also be made of sensors operating according to other physical principles, the measurement variables of which change depending on the load distribution acting on the bridge, for example, optical, capacitive or inductive sensors, which can detect a deformation of the bridge depending on the weight load distribution. In principle, use can also be made of piezoelectric force sensors. However, generally these are only suitable for detecting dynamic processes. For detecting a static weight load distribution or the position of the center of gravity transversely to the bed, active piezoelectric transducers are rather unsuitable due to their drift. However, detecting changes in the weight load distribution is possible.

In one embodiment of the bridge with only one weight sensor, the latter is preferably arranged in such a way that it, where possible, only detects the bearing force of the transverse element on the one side of the frame. Depending on the position of the center of gravity in the pelvic region of the borne person, the weight load distribution in the bridge changes between the two lateral support regions on the frame. The measurement variable detected by the sensor therefore changes when the position of the center of gravity in the pelvic region of the person to be monitored changes. In order to detect and evaluate the measurement variable, the sensor is connected to a controller. Both the upper rigid plate or the rigid plate portions and also the elastic bearing plate are made of metal, preferably of stainless steel. In order to increase the rigidity, the upper plate or the upper plate portions may comprise longitudinal edges, which are folded or bent by 90°, and/or reinforcement ribs extending in the longitudinal direction. The latter could be formed on the plate itself, for example by deep drawing. Alternatively, or in addition thereto, reinforcement elements can also, in the form of elongate profiles made of plastics or other materials, be adhesively bonded onto the plate or connected to the plate in another suitable manner.

In alternative embodiments of the bridge, further support regions can additionally be formed between the support regions at the outer ends of the plate, which further support regions are directly or indirectly mounted on the frame or the support device, for example supported by means of support elements on an elastic bearing plate arranged therebelow, on a bed slat or a transverse connection of the bed frame or the lying area.

In a further advantageous embodiment, the upper plate of the bridge comprises several—e.g. two or three—conjoined rigid portions. Adjacent plate portions can be connected, e.g. in a hinged or generally flexible manner by bands, by riveting, welding, adhesive bonding or in any other way. Additional sensors can be arranged in the region of the connection points of adjacent plate portions. Such bridges can also be placed in the case of beds with arced lying areas, for example onto a bed slat or onto two adjacent bed slats of a slatted frame, and adapt to the shape of these lying areas. Alternatively, it would also be possible in the case of an integral plate to form flexible zones between portions with high rigidity by virtue of the plate for example having a reduced strength and/or no reinforcing elements that increase the rigidity at those locations.

Optionally, the plates or the plate portions can comprise notches or indentations at the support or bearing regions, which expose springboard-like, slightly elastically deformable tongues, which are connected to the adjoining plate regions only via narrow connecting webs. Sensors in the form of strain gauges are arranged in the region of these connecting webs. In principle, respectively one or more support regions of the metal plate can be supported on a common connection means, which itself in turn is mounted on the frame. The connection means serves for force transmission between the bridge or the upper plate and the frame. In alternative embodiments, one or more sensors can also be formed on a connection means itself or between the connection means and the bridge or the plate or between the connection means and the frame.

The measuring device with the bridge embodied according to the invention is portable and can be configured in such a way that it can be applied both in the case of different beds and in the case of mattresses lying on the floor and that no tools whatsoever are required during the installation. Thanks to a low installation height of preferably less than 20 to 25 mm, the bridge can be placed between mattress and lying area of the bed frame in various beds, without interfering. The low width of the bridge enables the use in different beds. The measuring device can comprise a sheath made of hydrophobic plastic as a spray water protection, without impairing the function.

In a further alternative embodiment of the invention, the bridge can comprise one or more flexible sections, wherein these are e.g. spanned in a frame between the longitudinal struts, analogous to the covering of a patio lounger, and comprise sensors which can be tensioned. In such an embodiment, the sensors are preferably strain gauges which can be tensioned, in the edge region of the covering or of the bridge. The term “support regions” should accordingly be interpreted broadly. It comprises not only the zones of regions or a rigid bridge, which are supported directly or indirectly on the frame, but, in general, those zones or regions of a bridge or of the connection between the bridge and the frame, at which forces or torques for supporting the bridge are transmitted to the frame.

In the region of the bridge, the controller for the measuring device preferably comprises evaluation electronics with an analog/digital transducer device for detecting and digitizing the sensor signals and, in a separate external housing, a control part for evaluating the sensor signals and outputting alarm signals. Alternatively, the sensor signals can, for example, also be processed further directly by the control part at the bridge and evaluated there, for example by means of a processor arranged there. The controller is preferably configured in such a way that it can store the time profile of the sensor signals. Alternatively, it is also possible to store information derived from processing the measurement variable. Thus, the controller can, for example, comprise processing prescriptions, which filter out movements of the person, which are required to prevent this person having bedsores, from the measurement variable. Preferably, it is only movements with a predeterminable minimum impact that are detected as relevant movements. Here, additional criteria such as the behavior over time or accelerations may be taken into account.

If the frequency and intensity of the movements do not correspond to specific comparison criteria prescribed in the controller, the controller emits an alarm signal. Preferably, individual reference intervals can be predetermined in the controller for each person to be monitored depending on the respective physical condition. If the duration between two successive relevant movements of a person to be monitored exceeds the predetermined duration of such a reference interval, an alarm signal is emitted. The external control equipment of the controller preferably comprises a display with a green, a yellow and a red display light, corresponding to traffic lights. If the duration without detected relevant movements exceeds the first reference value, the display changes from green to yellow. If the duration without detected relevant movements exceeds the second reference value, the display changes from yellow to red. Additionally, or as an alternative thereto, it is also possible for acoustic alarm signals to be emitted and/or for alarm apparatuses in a patient room or at a central monitoring station to be activated by means of appropriate communication links.

The controller can additionally also be configured to detect reference variables, which can also be employed when processing the measurement variables. Thus, for example, measurement variables detected by the sensor or the sensors can be stored if the person lies in a defined position on the bed, particularly if the focus of the load of the bridge is approximately in the center of the bridge. Alternatively, or in addition thereto, the body weight of the person to be monitored or other parameters can also be provided to the controller. Together with these parameters and the measurement variable of the sensor, the controller can monitor not only the positional changes required for bedsore prophylaxis but also the position of the person in the transverse direction of the bed. Alternatively, or in addition thereto, the measurement variable can, for example, also be filtered in different manners or processed further in different manners, in order to obtain information about bodily functions therefrom, such as breathing or the pulse.

The measuring device preferably comprises two or more sensors. At least respectively one of these sensors is arranged in the region of the outer-most support regions of the bridge in such a way that it can detect the bearing force of the bridge on the frame in this region.

The bridge can comprise further sensors between the two outer-most support regions.

### BRIEF DESCRIPTION OF THE DRAWINGS

The invention will be described in more detail below on the basis of some figures. Here:

FIG. 1 shows a first arrangement of a bridge at the lying area of a bed,

FIG. 2 shows a detailed view of the arrangement from FIG. 1 in the region of transmission elements for supporting a bridge on a supporting element with sensors,

FIG. 3 shows a cross section of the arrangement from FIG. 1 in the region of a transmission element,

FIG. 4 shows a second arrangement of a bridge at the lying area of a bed,

FIG. 5 shows the bridge from FIG. 4 in a side view and in a plan view,

FIG. 6 shows a schematic depiction of the support and the measurement-variable detection in the case of bridges with one or more sensors,

FIG. 7 shows an arrangement of several bridges at the lying area of a bed,

FIG. 8 shows a further bridge in a perspective view, and

FIG. 9 shows a schematically depicted side view of the arrangement from FIG. 8.

### DETAILED DESCRIPTION OF THE PREFERRED EMBODIMENTS

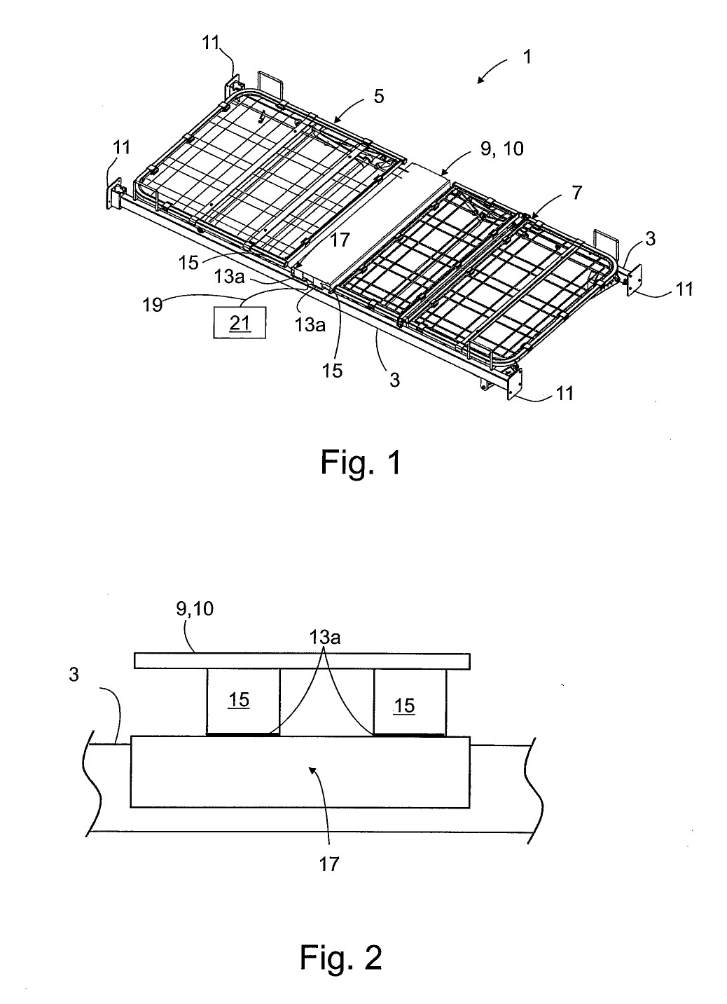

FIG. 1 shows a lying area 1, as is employed e.g. in the case of beds in hospitals and care homes for a mattress to be placed thereon. The lying area 1 comprises two parallel longitudinal struts 3, between which a back part 5 and a leg and foot part, abbreviated as foot part 7 below, are mounted in a pivotable manner. Formed on the ends of the longitudinal struts 3 are attachment plates 11 for attachment to a bed frame (not depicted)—which is also abbreviated as frame below. In alternative embodiments of the beds, the longitudinal struts 3 can also be embodied as a component of the frame which supports the lying area 1. Between the back part 5 and the foot part 7, the lying area 1 comprises a central part 9 in conventional beds, for example a rigid sheet-metal construction, which is generally securely connected to the two longitudinal struts 3 and cannot be moved relative thereto (not depicted). The central part 9 extends transversely to the bed, approximately in the region of the pelvis of a person lying in the bed.

According to the invention, this central part 9 is embodied as bridge 10 or it is complemented or replaced by such a bridge 10. This bridge 10 is a component of a measuring device with at least one sensor 13, the measurement variable of which can be changed depending on the load distribution acting on the bridge 10. The bridge 10 is configured as a force transmission means. As part of the lying area 1, it carries the weight applying a load thereon and is itself in turn supported by the longitudinal struts 3 or the bed frame.

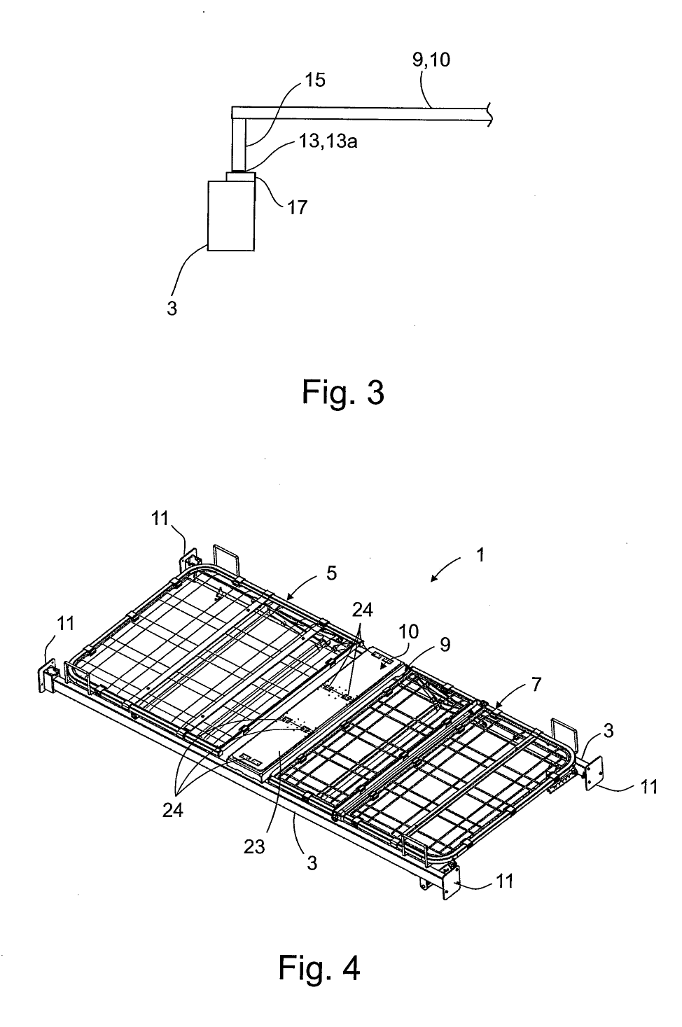

In a first embodiment in accordance with FIG. 1, the bridge 10 comprises a rigid metal plate folded over along the longitudinal edges thereof. In the region of each of the two narrow edges, this metal plate is supported on in each case one weighing area of a force sensor 13 by means of connection means in the form of in each case two e.g. cubic transmission elements 15. By way of example, the weighing areas can be embodied as resilient tongues of an L-shaped angled supporting element 17 attached to the respective longitudinal strut 3. FIG. 2 shows a detail of such an arrangement in a side view; FIG. 3 shows a cross section in the region of one of the transmission elements 15. The pressure or force sensor 13a is preferably attached in the transmission region between the bridge 10 and the longitudinal strut 3 in such a way that it has maximum sensitivity in relation to loads on the bridge 10. Depending on the type of force sensor 13a, it is therefore possible e.g. for a point with a relatively large elastic deformation or else a point with a relatively large bearing pressure to be more advantageous. The force sensor 13a or, in general, the sensor 13 can, in principle, be arranged at different points of the force transmission path between the load supported by the bridge 10 and the support device or the longitudinal strut 3, for example on the bridge 10 itself, on one of the connection means, in particular on the transmission element 15, on the carrier element 17 on the longitudinal strut 3 or between two adjacent parts in the force transmission path.

The sensor or sensors 13 or force sensors 13a are connected to an electronic controller 21 by means of one or more connection lines 19, which controller evaluates the measurement variables and generates control variables for e.g. an optical and/or acoustic alarm function. The controller 21 or parts of this controller 21 can also be arranged on the bridge 10 or at one or more of the sensors 13, 13a. In particular, the sensors 13, 13a can comprise e.g. actuation and/or evaluation electronics such as e.g. an A/D transducer (not depicted). For actuation and/or evaluation purposes, a plurality of sensors 13, 13a can also comprise common electronics (not depicted). Preferably, at least part of the controller 21 is arranged in a housing which, depending on requirements, can be attached to different positions of a bed, for example at the head end or foot end of the bed frame. The controller 21 preferably comprises a display, an optical and/or acoustic warning apparatus, keys or other input means for influencing equipment functions and an interface, for example a plug connection for an Ethernet connection for communicating with other equipment (not depicted).

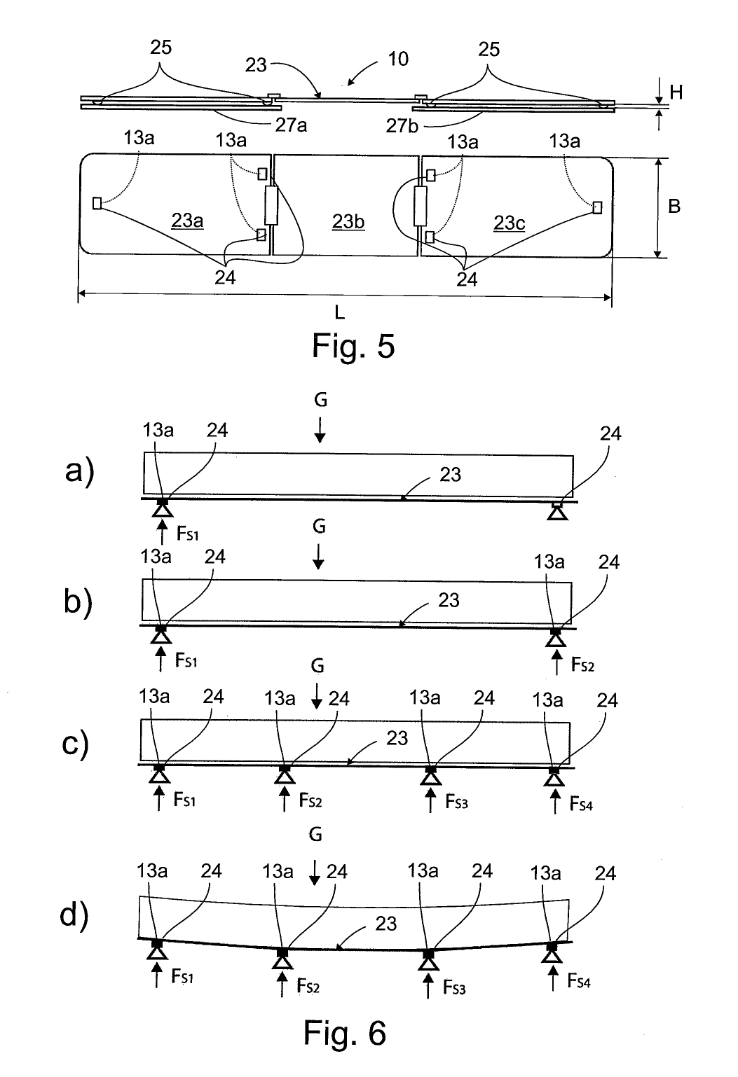

FIG. 4 shows an alternative embodiment of the bridge 10 at a lying area 1. This bridge 10 is depicted in a side view and in a plan view in FIG. 5. It comprises an approximately rectangular elongate plate 23, preferably made of metal, with a length L, which approximately corresponds to the width of the bed, and a width B, which, depending on embodiment, can lie in the range from approximately 5 cm to approximately 40 cm. By way of example, the width B of the plate 23 can be dimensioned such that it corresponds to the width of the central part 9 of the lying area 1. In alternative embodiments, the width B of the plate 23 can, for example, also correspond to the width of a bed slat of a slatted frame or the width of two or more bed slats including the space between these bed slats. By way of example, the plate 23 can be integral or made of a number of parts in the form of several portions 23a, 23b, 23c. The adjacent portions 23a, 23b, 23c are connected to one another. In order to produce these connections, different joining techniques such as e.g. welding, adhesively bonding or riveting can be employed. In particular, adjacent portions 23a, 23b, 23c can be connected to one another in a hinged manner by means of welded-on steel bands. As a result of different thicknesses and/or materials and/or overlapping regions of the portions 23a, 23b, 23c, mechanical properties of the bridge 10, such as e.g. the rigidity thereof, can be adapted in accordance with the respective requirements. At least in the region of the two narrow plate ends, T-shaped, springboard-like structures can be cut into the plate 23, preferably by means of a laser. These structures form support points or support regions 24, at which the plate 23 can be supported or carried directly or indirectly, by means of suitable connection means, on the bed frame or on the longitudinal struts 3. In each of these support regions 24, which can perform a slight elastic deformation in the case of load being applied, a strain gauge is arranged as a force sensor 13a. The bridge 10 depicted in FIGS. 4 and 5 comprises four additional support regions 24 in addition to the support regions 24 at the two ends. Of these, respectively two are arranged approximately at the end of the two outer thirds of the whole plate length L, i.e. in the vicinity of the respectively adjoining portions 23a, 23b, 23c of the plate 23. The three left-hand support regions 24 are each connected to a first contact plate 27a by means of a spacer 25 or are each supported on this first contact plate 27a. This first contact plate 27a is preferably aligned parallel to the plate 23 at a small distance H, which, for example, can lie in the millimeter range. In an analogous manner, the three right-hand support regions 24 are also connected to a second contact plate 27b or supported on this second contact plate 27b. The contact plates 27a, 27b have the effect of connection means, which can be employed to support the plate 23 or the bridge 10 on the support device or on a transverse connection connected to the support device or on bed slats. As an alternative or in addition to the support points 24 of the plate 23, it is also possible for the spacers 25 to comprise resilient structures and to be embodied with strain gauges as force sensors 13a. In the example depicted in FIG. 4, the bridge 10 lies on the central part 9, which is embodied as a rigid connection plate between the longitudinal struts 3, on the lying area 1. Since the bridge can simply be placed onto the connection plate, no tools whatsoever are required for the installation thereof. As a result of the low installation height thereof, which is generally less than 3 cm, preferably approximately 1 cm to 1.5 cm, the bridge is not in any way an impediment. Since the mattress now lies on the bridge 10, this bridge 10 can be considered to be an integral component of the lying area 1 or as bearing on the lying area 1. The bridge 10 in accordance with FIG. 5 can also simply be placed onto two adjacent slats of a slatted frame, which, without load, have convex or concave arcing. As a result of the hinged connection of the portions 23a, 23b, 23c, the plate 23 is adapted to the contour of the slats in such a way that the contact plates 27a, 27b of the two outer sections 23a, 23c lie on the slats, while the plate 23 itself does not touch the slats in any of the portions 23a, 23b, 23c. Depending on how it is viewed, the bridge 10 can only comprise the plate 23 with the support regions 24 or, alternatively, additionally also comprise the spacers 25 and the contact plates 27a, 27b. In the first case, the spacers 25 and the contact plates 27a, 27b are part of the connection means, by means of which the bridge 10 can be supported on the longitudinal struts 3 of the support device.

For improved understanding, FIG. 6 shows different arrangements of plates 23 with two or more support regions 24 and with one or more force sensors 13a arranged at such support regions. In the arrangement a), the plate 23 is supported in the region of its narrow-side ends on two support regions 24, but only comprises a sensor at the left-hand support region 24. The weight G applying a load to the plates 23 is supported at both support regions 24, with the force being applied to the left-hand support region 24 being denoted by Fₛ₁. In the arrangement b), a force sensor 13a is additionally arranged at the right-hand support region 24, which additional force sensor detects the associated support force Fₛ₂. In the arrangements c) and d), the plate 23 comprises three portions 23a, 23b, 23c connected to one another in a hinged manner and four support regions 24 arranged distributed over the length of the plate 23. Arranged at each of these support regions is a force sensor 13a, which records the respectively associated support force Fₛ₁, Fₛ₂, Fₛ₃, Fₛ₄. In the arrangement c), the support regions 24 lie in a plane. By contrast, in the arrangement d), the plate 23 lies on an uneven bearing layer, for example on flexible bed slats. The position of the individual support regions 24 of the plate 23 adapts to the shape of this bearing layer, and so the support regions 24 no longer lie in a common plane.

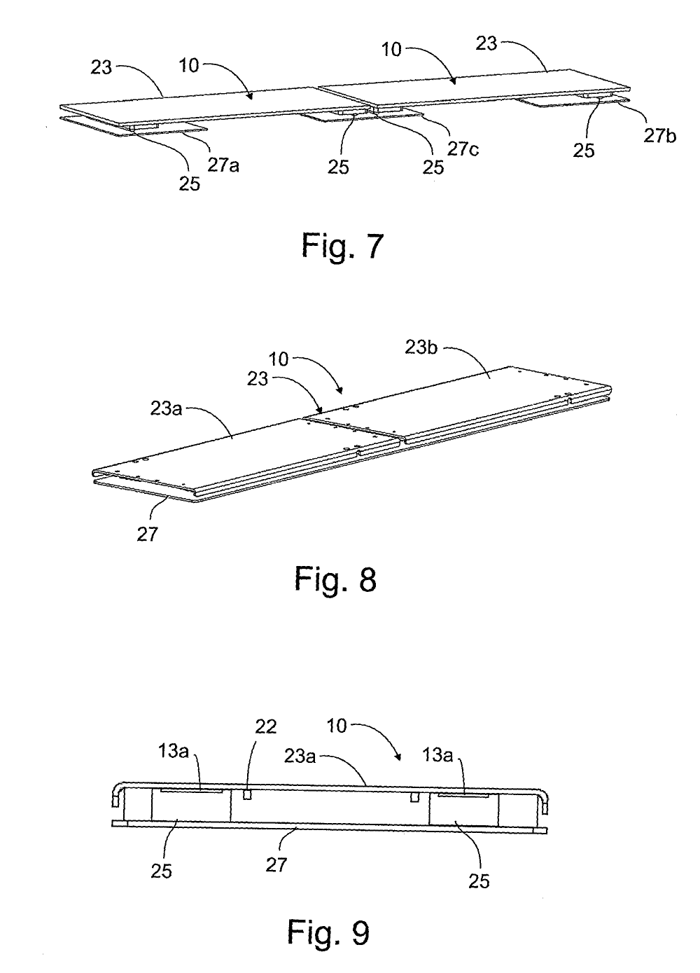

FIG. 7 shows an arrangement of two bridges 10, in which the adjacent ends of the plates 23 are supported indirectly on the bed frame by means of a central contact plate 27c (not depicted). The two outer contact plates 27a, 27b support the two plates 23 directly or indirectly on the longitudinal struts 3 or on the support device, analogously to the embodiment in accordance with FIG. 5.

FIG. 8 shows a particularly advantageous embodiment of a bridge 10, wherein the metal plate comprises two portions 23a, 23b, in which respectively one narrow strip of approximately 5 to 15 mm width is bent by 90°, or folded over, in the region of both longitudinal edges. The profile with the U-shaped cross section increases the rigidity of these plate portions 23a, 23b. Under the plate portions 23a, 23b, a bearing or contact plate 27 extends over the whole length of the bridge. The contact plate 27 is dimensionally stable but has a significantly higher bending elasticity than the portions 23a, 23b of the upper plate 23.

FIG. 9 schematically shows the arrangement from FIG. 8 in a side view. The portions 23a, 23b are supported on the contact plate 27 by means of support bodies or spacers 25. The force sensors 13a are arranged between the spacers 25 and the respective upper plate portion 23a, 23b. The spacers 25 are connected to the respective upper plate portion 23a, 23b and preferably lie only loosely on the contact plate 27. This arrangement is packaged in a protective sheath (not depicted). In the case of loosely arranged plates 23, plate portions 23a, 23b, 23c and/or contact plates 27, the protective sheath also keeps these elements together. The interior of the protective sheath is adapted to the dimensions of the bridge 10 in terms of shape and size. In particular, the protective sheath can comprise an opening and/or internal pockets, which can be closed by means of a hook-and-loop fastener or zip fastener (not depicted), for holding and positioning plate portions 23a, 23b, 23c. In addition to the bent longitudinal edges, the plate portions 23a, 23b comprise further elements which increase the rigidity, in the form of longitudinal ribs 22, which are adhesively bonded to the lower surfaces of the plate portions 23a, 23b. Even in the case of spacers with low heights, it is possible to produce bridges 10, the rigid plate portions 23a, 23b of which do not touch the lower contact plates 27, even when placed on arced lying areas.

1. A measuring device for detecting positional changes of persons in a bed, comprising a lying area (1) supported by a support device with a mattress lying thereon, at least one elongate bridge (10) supports the mattress and transmits a supported load to the support device, the at least one elongate bridge (10) comprises at least one plate (23) with at least one rigid portion (23a, 23b, 23c), which has a rigid design or increased rigidity, and at least one sensor (13, 13a) is arranged in a force transmission path between the supported load and the support device, that detects a measurement variable, which can be influenced by a load distribution of the supported load.

2. The measuring device as claimed in claim 1, wherein the sensor (13, 13a) or at least one of the sensors (13, 13a) is a force sensor (13a) to which at least one of pressure or tension is applied, or a strain gauge.

3. The measuring device as claimed in claim 2, the bridge (10) comprises at least two support regions (24) spaced apart from one another, wherein the support regions (24)
a) are supported directly by the support device, and the at least one sensor (13, 13a) is arranged by at least one of the support regions (24) on the bridge (10), or on the support device, or between the bridge (10) and the support device, or
b) is supported indirectly by the support device by connection elements, and the at least one of sensor (13, 13a) is arranged by the support regions (24) on the bridge (10), or on the connection elements, or between the bridge (10) and the connection elements, or between the connection elements and the support device, or on the support device.

4. The measuring device as claimed in claim 3, wherein at least one of the support regions (24) of the plate (23) is supported directly or indirectly on the support device by a spacer (25), the support region (24) or the spacer (25) comprises a resilient structure, and the at least one sensor (13, 13a) is arranged on the plate (23) or on the spacer (25) at said resilient structure.

5. The measuring device as claimed in claim 3, wherein the plate (23) comprises two or more rigid portions (23a, 23b, 23c), and respectively adjacent ones of the rigid portions (23a, 23b, 23c) are connected to one another in a hinged or flexible manner or arranged loosely next to one another.

6. The measuring device as claimed in claim 5, wherein at least two of the support regions (24) that are spaced apart from one another are formed on at least one of two outer-most ones of the rigid portions (23a, 23b, 23c) of the plate (23), the support regions (24) of said rigid portion (23a, 23b, 23c) are supported by spacers (25) on a common contact plate (27a, 27b) assigned to the respective rigid portion (23a, 23b, 23c), and the support regions (24) or the spacers (25) comprise resilient structures with the sensors (13, 13a).

7. The measuring device as claimed in claim 3, wherein the support regions (24) are supported indirectly by the support device by connection elements, at least one of the connection elements is a supporting element (17) which is attachable to the supporting device, and at least one sensor (13, 13a) is arranged on the supporting element (17).

8. The measuring device as claimed in claim 1, wherein the bridge (10) has a width B in a range from 5 cm to 40 cm, and an installation height of the bridge (10) is less than 3 cm.

9. The measuring device as claimed in claim 1, wherein the at least one sensor (13, 13a) is connected to a controller (21), said controller (21) is arranged wholly or partly in a housing that is independently placeable of the bridge (10) or wholly or partly on the bridge (10), and the controller (21) is configured for detecting and evaluating measurement variables of the at least one of sensor (13, 13a), including at least one of detecting a position of a center of gravity or changes in the center of gravity of a weight distribution applying a load to the bridge (10), and at least one of an optical, acoustic, or electric indicator for movement activity for estimating a risk of bedsores.

10. The measuring device as claimed in claim 9, wherein the controller (21) comprises a communication interface.

11. A bed with the measuring device as claimed in claim 1, wherein the lying area (1) includes a back part (5), a central part (9) and a foot part (7), and the bridge (10) is formed or arranged on the back part (5) or on the central part (9) of the lying area (1).

12. A method for processing measurement variables in the case of a bed as claimed in claim 11, wherein a relative position of a center of gravity of a weight applying a load to the bridge (10) is established based on measurement variables of the at least one sensor (13, 13a), values of at least one of the position of the center of gravity or the measurement variables of the at least one sensor (13, 13a) are stored periodically, changes in the position of the center of gravity are monitored, an insufficient movement activity is established by comparison with stored criteria, and an alarm is triggered in case of insufficient movement activity.
